(19) United States
(12) Patent Application Publication
(10) Pub. No.: US 2024/0060029 A1
(43) Pub. Date: Feb. 22, 2024

(54) CELL CULTURE APPARATUS AND CELL CULTURE METHOD

(71) Applicants: CANON MEDICAL SYSTEMS CORPORATION, Otawara-shi (JP); Canon Kabushiki Kaisha, Tokyo (JP)

(72) Inventor: Katsuaki DEGUCHI, Otawara (JP)

(73) Assignees: CANON MEDICAL SYSTEMS CORPORATION, Otawara-shi (JP); Canon Kabushiki Kaisha, Tokyo (JP)

(21) Appl. No.: 18/451,466

(22) Filed: Aug. 17, 2023

(30) Foreign Application Priority Data
Aug. 18, 2022 (JP) 2022-130596

(51) Int. Cl. C12M 1/00; C12M 1/12; C12M 3/04; C12M 1/36

(52) U.S. Cl. CPC C12M 29/00; C12M 25/10; C12M 27/10; C12M 41/48

(57) ABSTRACT

A cell culture apparatus of an embodiment includes a culture medium housing part, a cell housing part, a discharge path, a culture part, and a movable wall. The culture medium housing part houses a culture medium. The cell housing part houses the cells. The discharge path is the path of the waste liquid. The culture part has a hollow tubular shape with holes provided at both ends. The culture medium housing part, the cell housing part, and the discharge path are connected to one of the holes in a switchable manner. The movable wall is arranged in a slidable manner along an inner peripheral face of the culture part. The cell culture apparatus performs sucking from at least either the culture medium housing part or the cell housing part into the culture part and discharge from inside the culture part to the discharge path.

### CROSS-REFERENCE TO RELATED APPLICATIONS

This application is based upon and claims the benefit of priority from Japanese Patent Application No. 2022-130596, filed on Aug. 18, 2022, the entire contents of which are incorporated herein by reference.

### FIELD

Embodiments described herein relate generally to a cell culture apparatus and a cell culture method.

### BACKGROUND

Stem cells such as induced pluripotent stem cells (iPS cells) and embryonic stem cells (ES cells) have the ability to differentiate into cells with various functions and are expected to be applied in the field of regenerative medicine, the field of drug discovery, and the like.

Conventionally, when cells such as iPS cells and ES cells are cultured, proliferated cells are periodically detached from a culture area, and some of the detached cells are seeded into a new culture vessel to maintain the cells. In this process, a technique is known to adjust the bottom area of the culture vessel so that the cell density of the cells to be cultured is an appropriate cell density corresponding to the cells' ability to proliferate.

### BRIEF DESCRIPTION OF THE DRAWINGS

FIG. 1 is a schematic diagram of an example of the configuration of a cell culture apparatus according to an embodiment;

FIG. 2 is a flowchart of an example of processing executed by a control apparatus of the cell culture apparatus according to the embodiment;

FIG. 3 is a flowchart of an example of the processing executed by the control apparatus of the cell culture apparatus according to the embodiment;

FIG. 4 is a diagram illustrating an example of operations of the cell culture apparatus according to the embodiment;

FIG. 5 is a flowchart of an example of the processing executed by the control apparatus of the cell culture apparatus according to the embodiment;

FIG. 6 is a diagram illustrating an example of the operations of the cell culture apparatus according to the embodiment;

FIG. 7 is a flowchart of an example of the processing executed by the control apparatus of the cell culture apparatus according to the embodiment;

FIG. 8 is a diagram illustrating an example of the operations of the cell culture apparatus according to the embodiment;

FIG. 9 is a flowchart of an example of the processing executed by the control apparatus of the cell culture apparatus according to the embodiment;

FIG. 10 is a diagram illustrating an example of the operations of the cell culture apparatus according to the embodiment;

FIG. 11 is a flowchart of an example of the processing executed by the control apparatus of the cell culture apparatus according to the embodiment;

FIG. 12 is a diagram illustrating an example of the operations of the cell culture apparatus according to the embodiment;

FIG. 13 is a diagram illustrating an example of the operations of the cell culture apparatus according to the embodiment;

FIG. 14 is a flowchart of an example of the processing executed by the control apparatus of the cell culture apparatus according to the embodiment;

FIG. 15 is a diagram illustrating an example of the operations of the cell culture apparatus according to the embodiment;

FIG. 16 is a flowchart of an example of the processing executed by the control apparatus of the cell culture apparatus according to the embodiment;

FIG. 17 is a diagram illustrating an example of the operations of the cell culture apparatus according to the embodiment;

FIG. 18 is a flowchart of an example of the processing executed by the control apparatus of the cell culture apparatus according to the embodiment; and

FIG. 19 is a diagram illustrating an example of the operations of the cell culture apparatus according to the embodiment.

### DETAILED DESCRIPTION

A cell culture apparatus of an embodiment includes a culture medium housing part, a cell housing part, a discharge path, a culture part, and a movable wall. The culture medium housing part houses a culture medium for culturing cells. The cell housing part houses the cells. The discharge path is a path discharging reagents and the like related to cell culture. The culture part has a hollow tubular shape with holes provided at both ends. The culture medium housing part, the cell housing part, and the discharge path are connected to one of the holes in a switchable manner. The movable wall is inserted into the culture part from another hole of the culture part and is arranged in a slidable manner along an inner peripheral face of the culture part. The cell culture apparatus performs sucking from at least either the culture medium housing part or the cell housing part into the culture part and discharge from inside the culture part to the discharge path through the sliding of the movable wall.

The following describes embodiments of a cell culture apparatus and a cell culture method in detail with reference to the accompanying drawings. The cell culture apparatus and the cell culture method according to the present application are not limited by the embodiments shown below. The embodiments can be combined with other embodiments and conventional technologies to the extent that there is no contradiction in description. In the following description, similar components are denoted by common symbols, and duplicated descriptions are omitted.

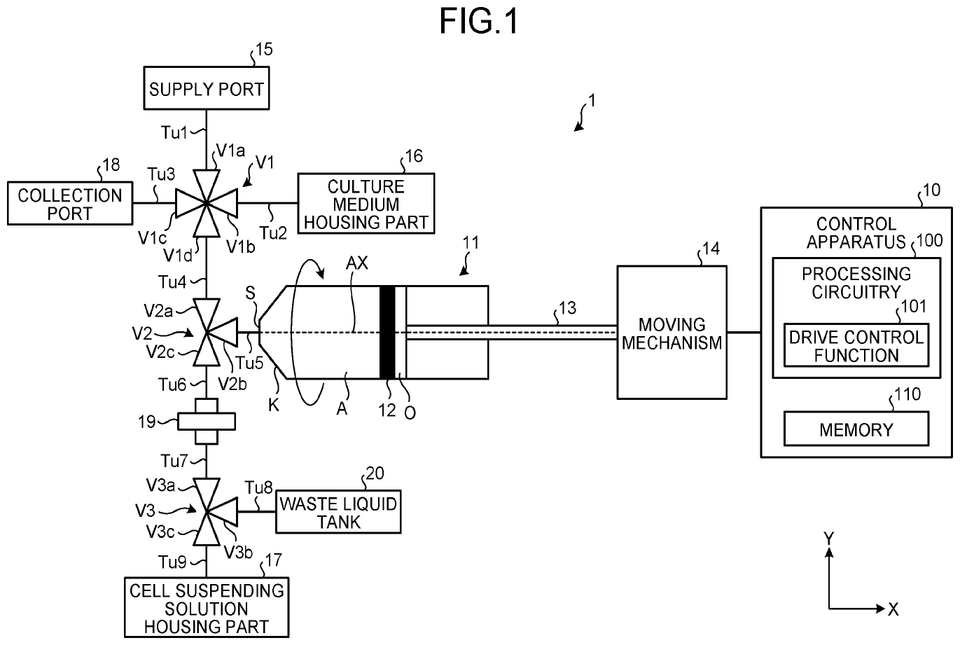

The following first describes the configuration of a cell culture apparatus 1 according to the present embodiment. The cell culture apparatus 1 is an apparatus automatically culturing cells while maintaining an aseptic condition inside. FIG. 1 is a schematic diagram of an example of the configuration of the cell culture apparatus 1 according to the present embodiment.

The cell culture apparatus 1 includes a control apparatus 10, a culture part 11, a movable wall 12, a piston 13, a moving mechanism 14, a supply port 15, a culture medium housing part 16, a cell suspending solution housing part 17, a collection port 18, a cell capturing part 19, a waste liquid tank 20, branches V1 to V3, and tubes Tu1 to Tu9.

The inside of the cell culture apparatus 1 according to the present embodiment (the inside of the culture part 11 and the tubes Tu1 to Tu9) is maintained at a positive pressure condition. This condition makes it difficult for outside air to enter the inside of the cell culture apparatus 1. The inside of the cell culture apparatus 1 is a closed system being not directly exposed to the outside air. This structure enables the cell culture apparatus 1 to maintain its inside aseptic condition.

The control apparatus 10 is an information processing apparatus comprehensively controlling the entire cell culture apparatus 1. The control apparatus 10 is connected to the moving mechanism 14 and the like. The configuration of the control apparatus 10 will be described below.

The culture part 11 adjusts its internal environment to perform culture of target cells. The culture part 11 has a hollow tubular shape with holes provided at both ends. An end of the culture part 11 provided with one hole is formed in a tapered shape with the diameter of the tubular shape increasing as the distance from the hole increases.

The culture part 11 is connected to the branch V2 by the tube Tu5. The culture part 11 is formed in a tapered shape as described above, and thus it has a sloped part K between a connection part S on which the tube Tu5 is mounted and a culture area A. The connection part S has a movable part and is capable of movement in the up-and-down direction in FIG. 1 and rotational movement and can tilt and rotate the culture part 11.

The part of the culture part 11 culturing the target cells is provided with the movable wall 12 and the piston 13. For example, a syringe can be used as the culture part 11. The culture part 11 is provided in a rotatable manner about an axis AX of the tubular shape. The culture part 11 rotates 360° with the axis AX as the axis of rotation during the culture of the target cells.

The movable wall 12 is inserted into the culture part 11 from another hole of the culture part 11 (the hole different from the hole provided on the side with the sloped part K) and is arranged in a slidable manner along an inner peripheral face of the culture part 11. In the present embodiment, the movable wall 12 is provided integrated with the piston 13. The movable wall 12 moves inside the culture part 11 in the x-axial direction (the positive direction and the negative direction) by the operation of the piston 13. Accordingly, the size of the culture area A performing the culture of the target cells can be changed, and thus the movable wall 12 can adjust the cell density of the target cells.

The piston 13 is inserted into the culture part 11 from the one hole of the culture part 11 and is arranged in a slidable manner along the inner peripheral face of the culture part 11. The piston 13 includes a push-in part O. The push-in part O is integrated with the movable wall 12. In FIG. 1, the movable wall 12 and the push-in part O are separate from each other, but the push-in part O may function as the movable wall 12.

The moving mechanism 14 controls the drive of each part of the cell culture apparatus 1. The moving mechanism 14 has a drive source such as a motor. For example, the moving mechanism 14 performs control to slide the movable wall 12 to perform sucking from at least either the supply port 15 or the culture medium housing part 16 into the culture part 11 and discharge from inside the culture part 11 to the waste liquid tank 20.

For example, the moving mechanism 14 is connected to a syringe pump (not illustrated) driving the piston 13. The moving mechanism 14 controls the syringe pump to drive the piston 13 and to perform control to draw and supply reagents and the like into the culture part 11. For example, the moving mechanism 14 is connected to a valve drive apparatus (not illustrated) opening and closing the valves of the branches V1 to V3. The moving mechanism 14 controls the valve drive apparatus to perform the opening and closing control of the valve.

The supply port 15 is a port for supplying the target cells suspended in a cell suspending solution, a detachment agent for detaching the target cells, and reagents such as a cleaning liquid. The supply port 15 is connected to a container housing the target cells and the reagents. The supply port 15 is connected to the branch V1 by the tube Tu1.

The target cells, the detachment agent, and the like in the housing container connected to the supply port 15 are drawn in a direction of the branch V2 by the drive of the piston 13 and are supplied to inside the culture part 11. This operation enables the cell culture apparatus 1 to automatically seed the target cells into the culture part 11 or detach the target cells in the culture part 11.

The culture medium housing part 16 houses a culture medium for culturing the target cells. The culture medium housing part 16 is connected to the branch V2 by the tube Tu2.

The cell suspending solution housing part 17 houses the cell suspending solution for suspending the target cells. The cell suspending solution housing part 17 is connected to the branch V3 by the tube Tu9.

The collection port 18 is a port for collecting the target cells. A collection container is connected to the collection port. The collection port 18 is connected to the branch V1 by the tube Tu3.

The cell capturing part 19 captures the target cells. The cell capturing part 19 captures the target cells and allows substances other than the target cells to pass through. The cell capturing part 19 is implemented by a selective membrane, a filter, or the like. The cell capturing part 19 is connected to the branch V3 by the tube Tu7.

The cell capturing part 19 captures only the target cells, and when the target cells in the culture part 11 are detached with the detachment agent, for example, even if the target cells mix with the detachment agent, by passing them through the cell capturing part 19, only the detachment agent can be flushed to the waste liquid tank.

The waste liquid tank 20 is a tank storing a waste liquid. The waste liquid tank 20 is connected to the branch V3 by the tube Tu8. For example, the culture medium, the detachment agent, and the like after use discharged from the culture part 11 pass through the branch V2, the cell capturing part 19, and the branch V3 to be stored in the waste liquid tank.

The branch V1 includes valves V1a, V1b, V1c and V1d. The valve V1a is connected to the supply port 15 by the tube Tu1. The valve V1b is connected to the culture medium housing part 16 by the tube Tu2. The valve V1c is connected to the collection port 18 by the tube Tu3. The valve V1d is connected to a valve V2a of the branch V2 by the tube Tu4.

The branch V2 includes valves V2a, V2b, and V2c. The valve V2a is connected to the valve V1d of the branch V1 by the tube Tu4. The valve V2b is connected to the culture part 11 by the tube Tu5. The valve V2c is connected to the cell capturing part 19 by the tube Tu6.

The branch V3 includes valves V3a, V3b, and V3c. The valve V3a is connected to the cell capturing part 19 by the tube Tu7. The valve V3b is connected to the waste liquid tank 20 by the tube Tu8. The valve V3c is connected to the cell suspending solution housing part 17 by the tube Tug.

Each valve of the branches V1 to V3 has a valve drive apparatus. The valve drive apparatus is connected to the moving mechanism 14. The drive apparatus of each valve uses the driving force of the moving mechanisms 14 to perform control to make each valve an open state or a closed state. A drive control function 101 controls the drive source such as a motor of the moving mechanism 14 to make each valve an open state or a closed state and to perform supply or discharge of the reagents and the like.

The following describes the configuration of the control apparatus 10. The control apparatus 10 has processing circuitry 100 and a memory 110. FIG. 1 illustrates only the processing circuitry 100 and the memory 110 in the control apparatus 10, but the control apparatus 10 may further include an input interface, a display, and the like.

The processing circuitry 100 controls the operation of the cell culture apparatus 1. For example, the processing circuitry 100 is implemented by a processor.

The processing circuitry 100 reads a computer program stored by the memory 110 and executes it to execute the drive control function 101.

The drive control function 101 controls the moving mechanism 14 to operate each part of the cell culture apparatus 1. For example, the drive control function 101 controls the moving mechanism 14 to drive the piston 13 and to draw the target cells to be cultured into the culture part 11. For example, the drive control function 101 controls the moving mechanism 14 to move the movable wall 12 by the force of the piston 13 and to perform supply, discharge, and the like of the reagents such as the culture medium.

For example, the drive control function 101 controls the moving mechanism 14 to rotate the culture part 11 at a certain speed during the culture of the target cells. This operation causes the drive control function 101 to prevent an imbalance from occurring in the cell density of the target cells in the culture part 11.

The target cells move inside the culture part 11 due to rotation, and thus even if the target cells in the culture medium are temporarily out of contact with the culture medium, the target cells will return to being in contact with the culture medium over time, thus preventing the drying of the target cells. Thus, the target cells can be cultured even without filling the culture part 11 with the culture medium, and the amount of the culture medium used can also be reduced.

The drive control function 101 controls the drive apparatuses of the respective valves of the branches V1 to V3 to perform control to open and close the valves. The details of control processing of each part by the drive control function 101 will be described below.

The memory 110 stores therein various types of data. The memory 110 is connected to the processing circuitry 100. For example, the memory 110 stores therein information on cell culture (for example, information on the type of the target cells, the type of the culture medium, the elapsed time from the start of culture, the elapsed time from the last culture medium replacement, and the like), control information such as the rotation rate of the moving mechanism 14, various computer programs for implementing various types of functions by being read and executed by the processing circuitry 100, and the like. For example, the memory 110 is implemented by a semiconductor memory element such as a random access memory (RAM) or a flash memory, a hard disk, an optical disk, or the like.

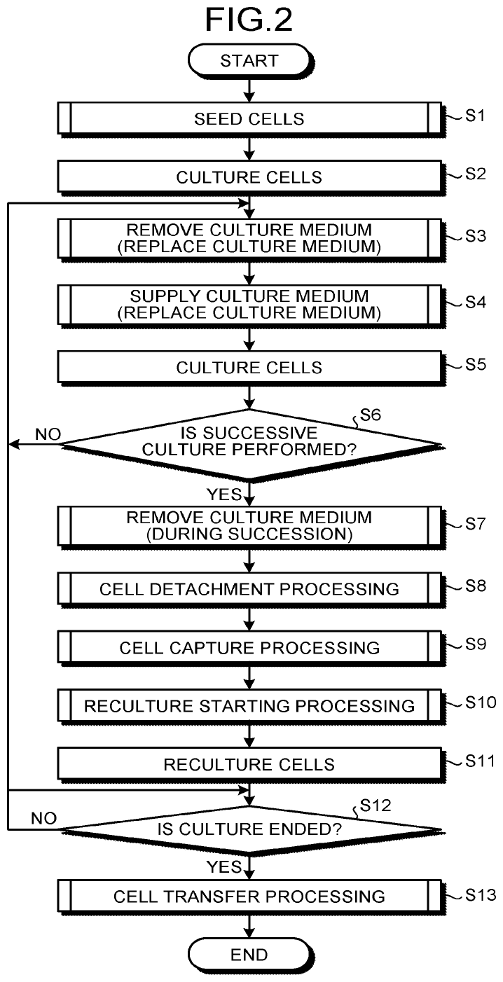

The following describes processing executed by the control apparatus 10 of the cell culture apparatus 1 according to the present embodiment. The following first describes an outline of the processing executed by the control apparatus 10 of the cell culture apparatus 1. FIG. 2 is a flowchart of an example of the processing executed by the control apparatus 10 of the cell culture apparatus 1. FIG. 2 is an example of the overall procedure of the processing executed by the control apparatus 10 of the cell culture apparatus 1.

First, the drive control function 101 controls the drive of the moving mechanism 14 to operate each part of the cell culture apparatus 1 and to seed target cells C (Step S1).

Next, the drive control function 101 cultures the target cells C for a certain period of time (Step S2). Next, the drive control function 101 performs processing of removing the culture medium for culture medium replacement (Step S3).

Next, the drive control function 101 performs processing of supplying the culture medium for culture medium replacement (Step S4). Next, the drive control function 101 cultures the target cells C for a certain period of time (Step S5). After a lapse of the certain period of time, the drive control function 101 determines whether to perform successive culture (Step S6). For example, the drive control function 101 determines whether to perform the successive culture based on whether the number of times the culture has been performed exceeds a preset threshold. If the successive culture is not performed (No at Step S6), the process moves to the processing at Step S3.

On the other hand, if the successive culture is performed (Yes at Step S6), the drive control function 101 performs processing of removing the culture medium for the succession (Step S7). Next, the drive control function 101 performs processing of detaching the target cells C from the culture part 11 (Step S8). Next, the drive control function 101 performs processing of capturing the target cells C by the cell capturing part 19 (Step S9).

Next, the drive control function 101 performs processing of starting reculture of the target cells C (Step S10). Next, the drive control function 101 recultures the target cells C for a certain period of time (Step S11). After a lapse of the certain period of time, the drive control function 101 determines whether to end the culture (Step S12). For example, the drive control function 101 determines whether to end the culture based on whether the number of times the reculture has been performed exceeds a preset threshold.

If the culture is not ended (No at Step S12), the process moves to the processing at Step S3. On the other hand, if the culture is ended (Yes at Step S12), the drive control function 101 performs transfer processing of transferring the target cell C to another process and ends this processing (Step S13).

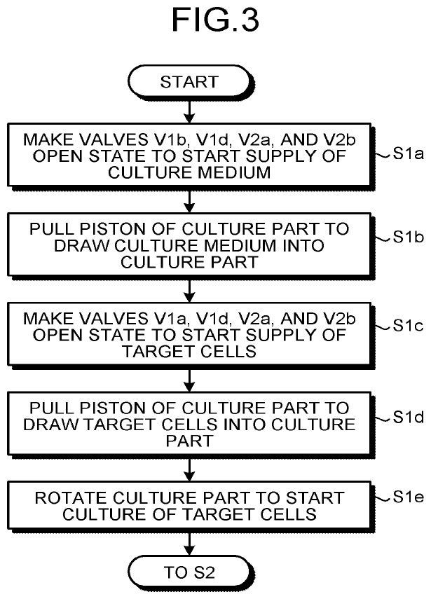

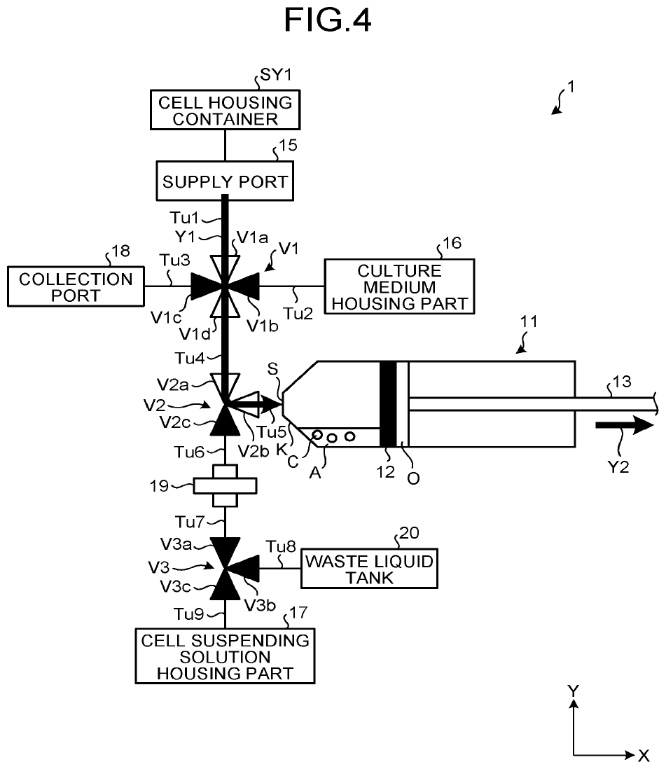

The following describes characteristic processing executed by the control apparatus 10 of the cell culture apparatus 1 according to the present embodiment. The following first describes cell seeding processing using FIG. 3 and FIG. 4. The cell seeding processing is processing corresponding to S1 in FIG. 2. FIG. 3 is a flowchart of an example of the processing executed by the control apparatus 10 of the cell culture apparatus 1. FIG. 4 is a diagram illustrating an example of operations of the cell culture apparatus 1.

As a precondition, it is assumed that a cell housing container SY1 housing the target cells C together with the cell suspending solution is connected to the supply port 15. The cell housing container SY1 in this case is an example of the cell housing part. In addition, it is assumed that all the valves are in a closed state.

First, the drive control function 101 makes the valves V1b, V1d, V2a, and V2b an open state to start supply of the culture medium to the culture part 11 (Step S1a). The amount of the culture medium supplied is set in advance in accordance with the type, amount, and the like of the target cells.

Next, the drive control function 101 pulls the piston 13 of the culture part 11 to draw the culture medium into the culture part 11 (Step S1b). In the present embodiment, after a certain amount of the culture medium is supplied to the culture part 11, the drive control function 101 makes all the valves an open state.

Next, the drive control function 101 makes the valves V1a, V1d, V2a, and V2b an open state to start supply of the target cells C to the culture part 11 (Step S1c). Next, the drive control function 101 pulls the piston 13 to draw the target cells C into the culture part 11 (Step S1d). Specifically, the drive control function 101 moves the piston 13 in a direction of the arrow Y1 in FIG. 2.

Accordingly, the target cells C are drawn in the cell housing container SY1 connected to the supply port 15 in a direction of the valve V1a. The drawn target cells C, together with the cell suspending solution, flow in a direction indicated by the arrow Y2 in FIG. 2 and pass through the tubes Tu1, Tu4, and Tu5 in this order to be supplied to the culture part 11.

In the present embodiment, after the target cells C are supplied to the culture part 11, the drive control function 101 performs control to make all the valves a closed state.

Next, the drive control function 101 rotates the culture part 11 to start culture of the target cells C (Step S1e). Specifically, the drive control function 101 rotates the culture part 11 with the axis AX of the tubular culture area A as the axis of rotation and moves to the processing at Step S2 in FIG. 2.

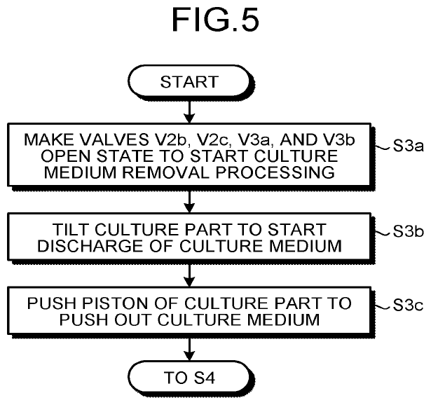

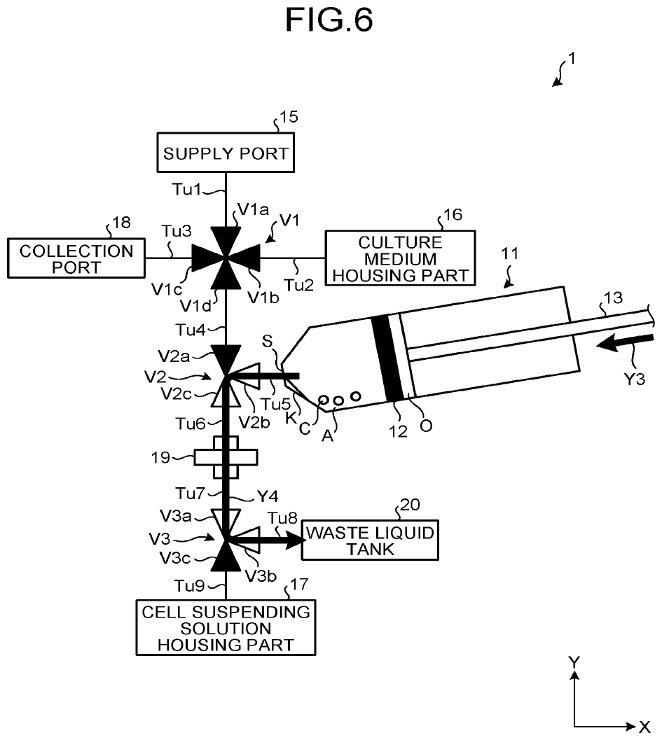

The following describes medium removal processing removing the culture medium from the culture part 11 in order to replace the culture medium using FIG. 5 and FIG. 6. The medium removal processing during culture medium replacement is processing corresponding to S3 in FIG. 2. FIG. 5 is a flowchart of an example of the processing executed by the control apparatus 10 of the cell culture apparatus 1. FIG. 6 is a diagram illustrating an example of operations of the cell culture apparatus 1. The culture medium removal processing for replacing the culture medium is performed after the culture of the target cells C has been performed for the certain period of time at Step S2 in FIG. 2.

First, the drive control function 101 makes the valves V2b, V2c, V3a, and V3b an open state to start the culture medium removal processing (Step S3a).

Next, the drive control function 101 tilts the culture part 11 at a certain angle and starts discharge of the culture medium (Step S3b). The certain angle is set in advance in accordance with the type of target cells C, the type of the culture medium, the amount of the culture medium, and force with which the piston 13 pushes out the culture medium, and the like. For example, the drive control function 101 specifies an angle at which the culture part 11 is tilted based on correspondence information stored in the memory 110 or the like and associating these pieces of information and the angle at which the culture part 11 is tilted with each other and tilts the culture part 11 at that angle.

Next, the drive control function 101 pushes the piston 13 to push out the culture medium (Step S3c). Specifically, the drive control function 101 moves the piston 13 in a direction of the arrow Y3 in FIG. 6 to push out the culture medium in the culture part 11 into the tube Tu5.

The movement of the piston 13 in this process is adjusted by the drive control function 101 so as not to crush the target cells C. The culture medium pushed out by the piston 13 flows in a direction indicated by the arrow Y4 and passes in order through the tubes Tu5 and Tu6, the cell capturing part 19, and the tubes Tu7 and Tu8 to be discharged into the waste liquid tank 20. The tubes Tu5, Tu6, Tu7 and Tu8 in this case are examples of the discharge path.

Even if the target cells C are pushed out together with the culture medium, for example, the target cells C are captured by the cell capturing part 19. The target cells C captured by the cell capturing part 19 can be returned to the culture part 11 by suspending them in the cell suspending solution, as described below.

In the present embodiment, the drive control function 101 performs control to make all the valves a closed state after the culture medium is discharged into the waste liquid tank 20. Subsequently, the drive control function 101 performs control to restore the tilt of the culture part 11 and moves to the processing at Step S4 in FIG. 2.

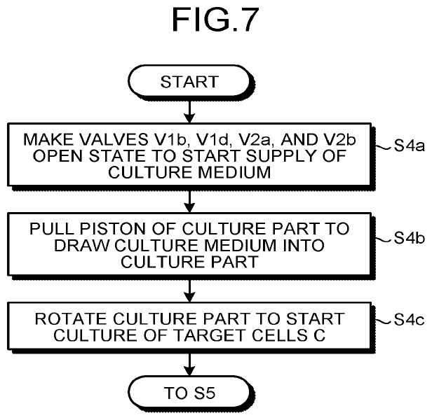

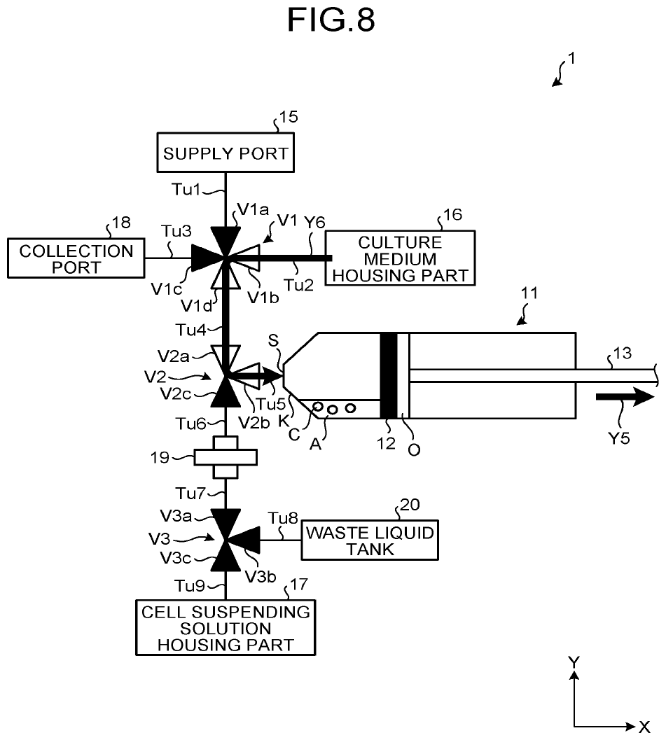

The following describes processing of supplying the culture medium to the culture part 11 in order to replace the culture medium using FIG. 7 and FIG. 8. Culture medium supply processing for replacing the culture medium is processing corresponding to S4 in FIG. 2. FIG. 7 is a flowchart of an example of the processing executed by the control apparatus 10 of the cell culture apparatus 1. FIG. 8 is a diagram illustrating an example of operations of the cell culture apparatus 1. The culture medium supply processing for replacing the culture medium is performed after the culture medium removal processing is performed at Step S3 in FIG. 2.

First, the drive control function 101 makes the valves V1b, V1d, V2a, and V2b an open state to start supply of the culture medium (Step S4a).

Next, the drive control function 101 pulls the piston 13 to draw the target cells C into the culture part 11 (Step S4b). Specifically, the drive control function 101 moves the piston 13 in a direction of the arrow Y5 in FIG. 8. Accordingly, the culture medium is drawn in the culture medium housing part 16 toward the valve V1b. The amount of the culture medium to be drawn is set in advance in accordance with the amount of the target cells to be cultured, the type of the target cells, the culture medium used for the culture, and the like. The amount of movement of the piston 13 is adjusted by the drive control function 101 in accordance with the amount of the culture medium, the amount of the target cells C, and the like.

The drawn culture medium flows in a direction indicated by the arrow Y6 in FIG. 8 and passes in order through the tubes Tu2, Tu4, and Tu5 to be supplied to the culture part 11. In the present embodiment, the drive control function 101 performs control to make all the valves a closed state after the culture medium is supplied to the culture part 11.

Next, the drive control function 101 rotates the culture part 11 to start culture of the target cells C (Step S4c). Subsequently, the drive control function 101 performs control to make all the valves a closed state and moves to the processing at Step S5 in FIG. 2.

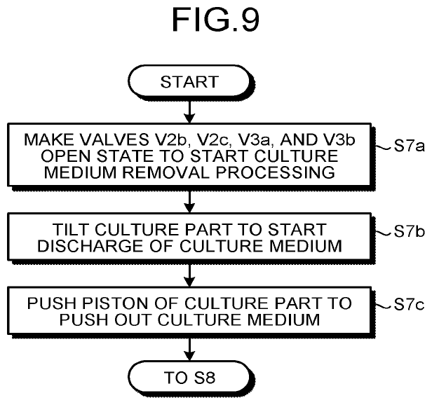

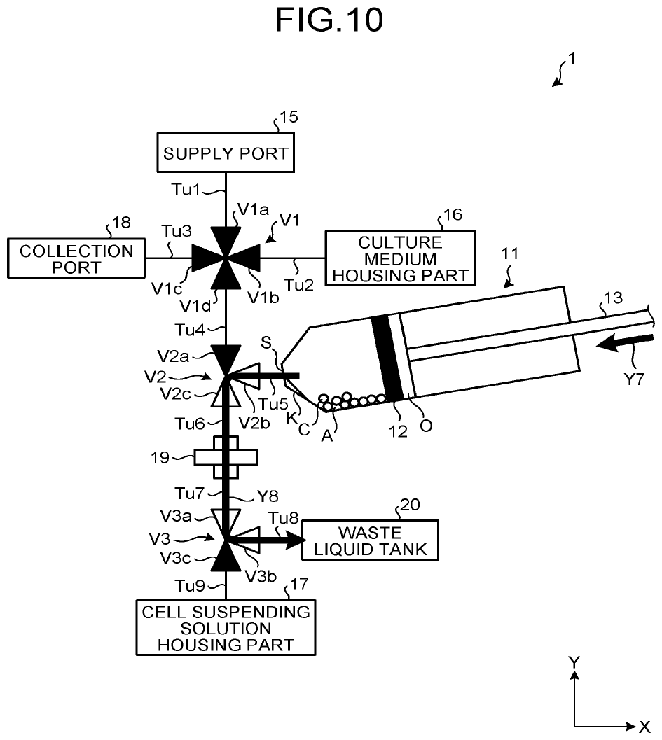

The following describes processing when the culture medium is removed from the culture part 11 in order to perform succession using FIG. 9 and FIG. 10. Culture medium removal processing during the succession is processing corresponding to S7 in FIG. 2. FIG. 9 is a flowchart of an example of the processing executed by the control apparatus 10 of the cell culture apparatus 1. FIG. 10 is a diagram illustrating an example of operations of the cell culture apparatus 1.

The culture medium removal for performing the succession is performed after the culture of the target cells C has been performed for the certain period of time with the culture medium replaced at Step S4 in FIG. 2. In the present embodiment, the succession refers to taking some of the cultured target cells C and performing culture again with the remaining target cells C.

First, the drive control function 101 makes the valves V2b, V2c, V3a, and V3b an open state to start the culture medium removal processing (Step S7a).

Next, the drive control function 101 tilts the culture part 11 at a certain angle and starts discharge of the culture medium (Step S7b). Next, the drive control function 101 pushes the piston 13 to push out the culture medium (Step S7c). Specifically, the drive control function 101 moves the piston 13 in a direction of the arrow Y7 in FIG. 10 to push out the culture medium in the culture part 11 into the tube Tu5.

The culture medium pushed out by the piston 13 flows in a direction indicated by the arrow Y8 and passes in order through the tubes Tu5 and Tu6, the cell capturing part 19, and the tubes Tu7 and Tu8 to be discharged into the waste liquid tank 20.

In the present embodiment, the drive control function 101 performs control to make all the valves a closed state after the culture medium is discharged into the waste liquid tank 20. Subsequently, the drive control function 101 performs control to restore the tilt of the culture part 11 and moves to the processing at Step S8 in FIG. 2.

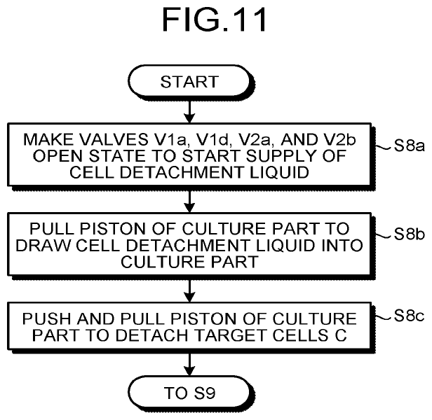

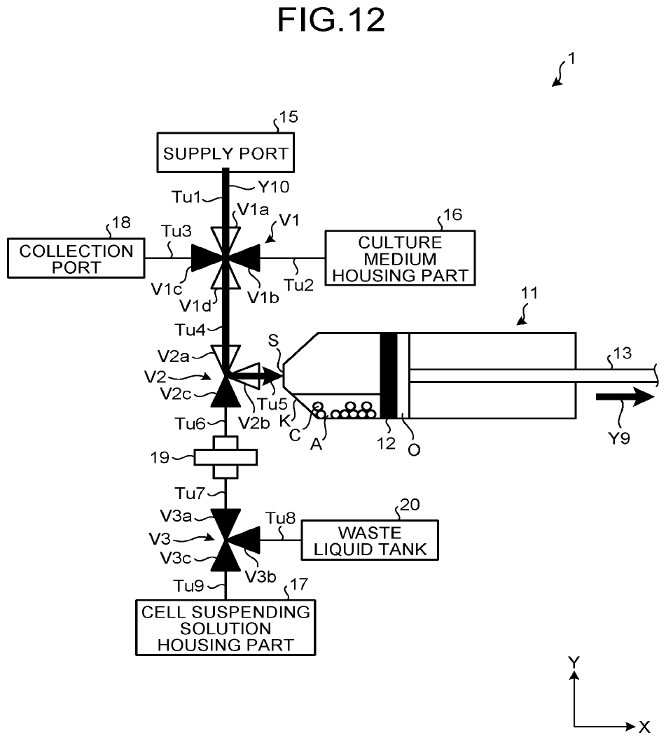

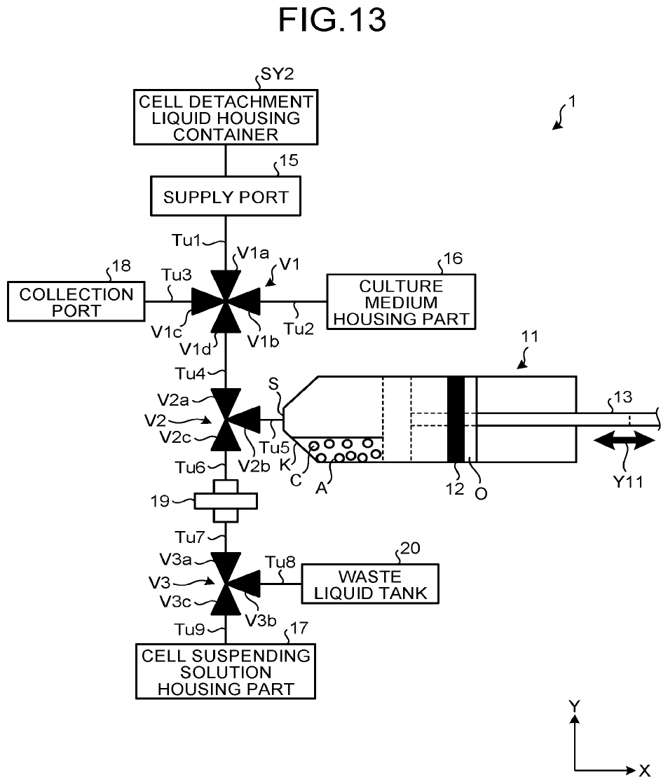

The following describes cell detachment processing detaching the target cells C from the culture part 11 in order to perform the succession using FIG. 11 to FIG. 13. The cell detachment processing is processing corresponding to S8 in FIG. 2. FIG. 11 is a flowchart of an example of the processing executed by the control apparatus 10 of the cell culture apparatus 1. FIG. 12 and FIG. 13 are diagrams illustrating examples of the operations of the cell culture apparatus 1. The cell detachment processing for performing the succession is performed after the culture medium removal processing is performed at Step S7 in FIG. 2.

When the cell detachment processing for performing the succession is performed, a cell detachment liquid housing container SY2 housing a cell detachment liquid is connected to the supply port 15. For example, a solution mainly containing enzymes with proteolytic activity can be used as the cell detachment liquid.

First, the drive control function 101 makes the valves V1a, V1d, V2a, and V2b an open state to start supply of the cell detachment liquid (Step S8a).

Next, the drive control function 101 pulls the piston 13 to draw the cell detachment liquid into the culture part 11 (Step S8b). Specifically, the drive control function 101 moves the piston 13 in a direction of the arrow Y9 in FIG. 12. Accordingly, the cell detachment liquid is drawn in the cell detachment liquid housing container SY2 connected to the supply port 15 toward the valve V1a. The amount of movement of the piston 13 is adjusted by the drive control function 101 in accordance with the amount of the cell detachment liquid to be supplied and the amount of the target cells C.

The drawn cell detachment liquid flows in a direction indicated by the arrow Y10 in FIG. 12 and passes in order through the tubes Tu1, Tu4, and Tu5 to be supplied to the culture part 11. In the present embodiment, the drive control function 101 makes all the valves a closed state after the cell detachment liquid is supplied to the culture part 11.

Next, the drive control function 101 pushes and pulls the piston 13 to detach the target cells C (Step S8c). Specifically, the drive control function 101 continuously reciprocates the piston 13 as indicated by the arrow Y11 in FIG. 13 after the supply of the cell detachment liquid.

Accordingly, the cell detachment liquid is stirred in the culture part 11. The culture part 11 has the sloped part K, and thus a flow is likely to occur in the internal cell detachment liquid by stirring. Thus, the cell culture apparatus 1 can easily detach the target cells C from the culture part 11.

In the present embodiment, the drive control function 101 performs control to make all the valves a closed state after the detachment of the target cells C and moves to the processing at Step S9 in FIG. 2.

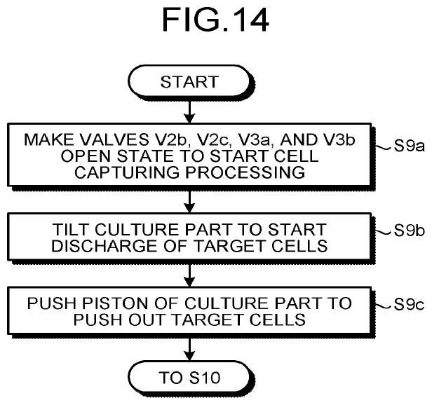

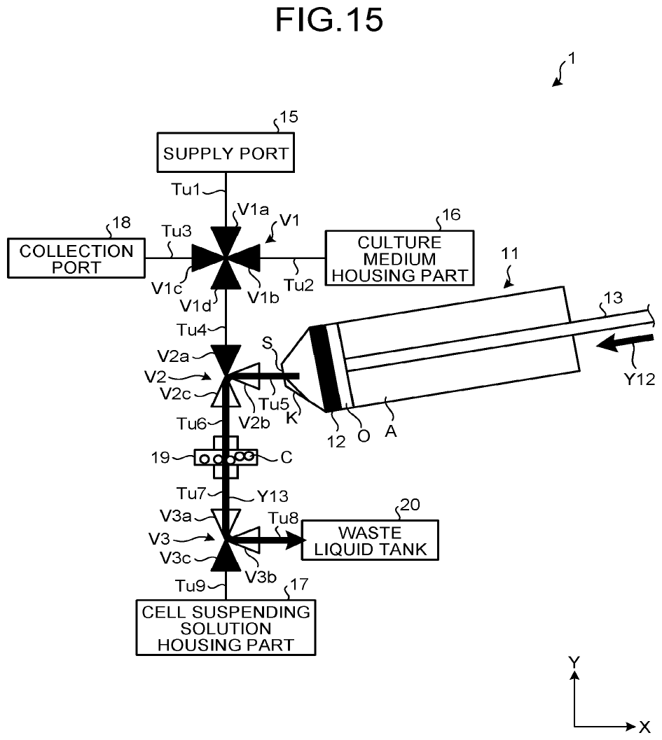

The following describes cell capturing processing capturing the target cells C in order to perform the succession using FIG. 14 and FIG. 15. The cell capturing processing is processing corresponding to S9 in FIG. 2. FIG. 14 is a flowchart of an example of the processing executed by the control apparatus 10 of the cell culture apparatus 1. FIG. 15 is a diagram illustrating an example of operations of the cell culture apparatus 1. The cell capturing processing for performing the succession is performed after the cell detachment processing is performed at Step S8 in FIG. 2.

First, the drive control function 101 makes the valves V2b, V2c, V3a, and V3b an open state to start the cell capturing processing (Step S9a).

Next, the drive control function 101 tilts the culture part 11 at a certain angle and starts discharge of the target cells C (Step S9b). The certain angle is set in advance in accordance with the type, amount, and the like of the target cells C. For example, the drive control function 101 specifies an angle at which the culture part 11 is tilted based on correspondence information stored in the memory 110 or the like and associating these pieces of information and the angle at which the culture part 11 is tilted with each other and tilts the culture part 11 at that angle.

Next, the drive control function 101 pushes the piston 13 to push out the target cells C (Step S9c). Specifically, the drive control function 101 moves the piston 13 in a direction of the arrow Y12 in FIG. 15 to push out the detached target cells C in the culture part 11 into the tube Tu5 together with the cell detachment liquid.

In this process, the drive control function 101 moves the piston 13 to a position closer to the branch V2 than that when removal of the culture medium is performed. The cell detachment liquid pushed out by the piston 13 flows in a direction indicated by the arrow Y13 and passes in order through the tubes Tu5 and Tu6, the cell capturing part 19, and the tubes Tu7 and Tu8 to be discharged into the waste liquid tank 20.

The target cells C pushed out from the culture part 11 together with the cell detachment liquid are captured by the cell capturing part 19. For the cell capturing part 19, one having properties corresponding to the type of the target cells C is used.

In the present embodiment, the drive control function 101 performs control to make all the valves a closed state after the cell detachment liquid is discharged into the waste liquid tank 20. The drive control function 101 performs control to restore the tilt of the culture part 11 and moves to the processing at Step 10 in FIG. 2.

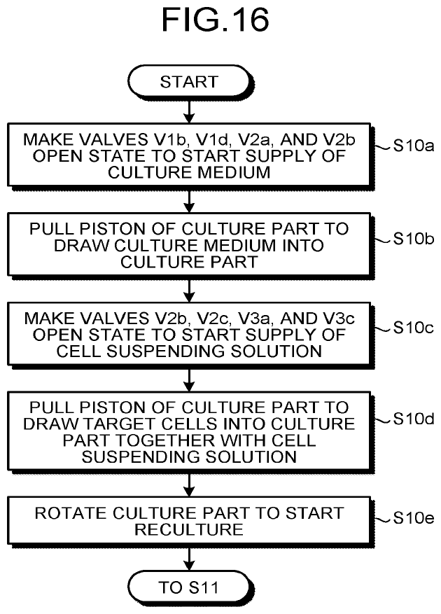

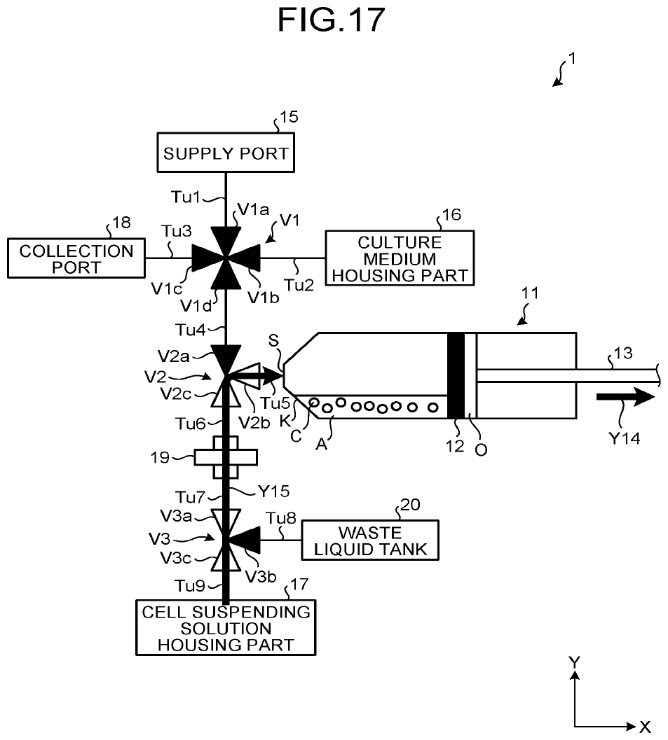

The following describes reculture starting processing returning the target cells C captured for performing the succession to the culture part 11 using FIG. 16 and FIG. 17. The reculture starting processing is processing corresponding to S10 in FIG. 2. FIG. 16 is a flowchart of an example of the processing executed by the control apparatus 10 of the cell culture apparatus 1. FIG. 17 is a diagram illustrating an example of operations of the cell culture apparatus 1. The reculture starting processing is performed after the cell capturing processing is performed at Step S9 in FIG. 2.

First, the drive control function 101 makes the valves V1b, V1d, V2a, and V2b an open state to start supply of the culture medium (Step S10a). The amount of the culture medium supplied during the succession is set in advance in accordance with the type and amount of the target cells C, the amount of the cell suspending solution for use in the reculture starting processing, and the like.

Next, the drive control function 101 pulls the piston 13 to draw the culture medium into the culture part 11 (Step S10b). In the present embodiment, after a certain amount of the culture medium is supplied to the culture part 11, the drive control function 101 makes all the valves an open state.

Next, the drive control function 101 makes the valves V1b, V2c, V3a, and V3c an open state to start supply of the cell suspending solution (Step S10c).

Next, the drive control function 101 pulls the piston 13 to draw the target cells C into the culture part 11 together with the cell suspending solution (Step S10d). Specifically, the drive control function 101 moves the piston 13 in a direction of the arrow Y14 in FIG. 17.

This operation draws the cell suspending solution in the cell suspending solution housing part 17 toward the valve V3c. The drawn cell suspending solution flows in a direction indicated by the arrow Y15 in FIG. 17 and passes in order through the tubes Tug and Tu7, the cell capturing part 19, and the tubes Tu6 and Tu5 to be supplied to the culture part 11.

In this process, the cell suspending solution suspends the target cells C captured in the cell capturing part 19. Thus, the target cells C will be supplied to the culture part 11 together with the cell suspending solution. The piston 13 has moved in the direction of the arrow Y14 in FIG. 17, whereby the size of the culture area A can be increased from that in the previous culture.

Thus, by increasing the size of the culture area A during the succession, it is possible to prevent the cell density of the target cell C from becoming too high in the successive culture, which is performed with an increased amount of the target cells C compared to that during the culture before the succession.

In the present embodiment, the drive control function 101 makes all the valves a closed state after the target cells C are supplied to the culture part 11. Next, the drive control function 101 rotates the culture part 11, starts reculture of the target cells C, and moves to the processing at Step S11 in FIG. 2 (Step S10e). Subsequently, the drive control function 101 repeats the processing at Steps S3 to S11 in FIG. 2. The target cells C are thereby repeatedly cultured and proliferated.

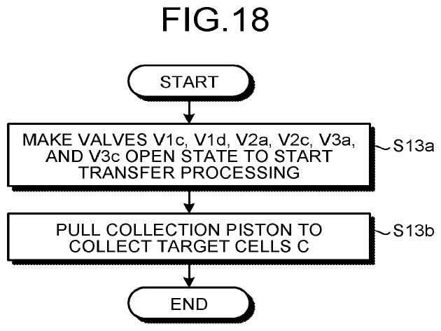

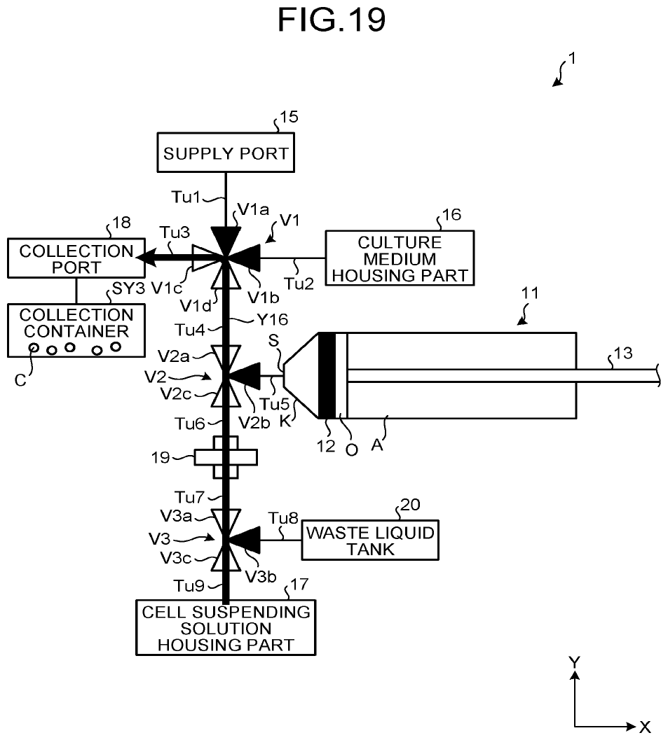

The following describes transfer processing when the culture is ended and the target cells C are transferred to another process such as an experiment using FIG. 18 and FIG. 19. The transfer processing is processing corresponding to S13 in FIG. 2. FIG. 18 is a flowchart of an example of the processing executed by the control apparatus 10 of the cell culture apparatus 1. FIG. 19 is a diagram illustrating an example of operations of the cell culture apparatus 1.

As a precondition, it is assumed that the cell capturing processing at Step S9 in FIG. 2 has been performed and the target cells C are captured in the cell capturing part 19. It is assumed that a collection container SY3 for collecting the target cells C is connected to the collection port 18.

It is assumed that a collection piston (not illustrated) different from the piston 13 is connected to the collection container SY3. In the present embodiment, it is assumed that the collection piston is movable in the x-axial direction (the positive direction and the negative direction) by a collection syringe pump (not illustrated) connected to the collection piston. The collection syringe pump is connected to the moving mechanism 14 and is driven by the control of the moving mechanism 14.

First, the drive control function 101 makes the valves V1c, V1d, V2a, V2c, V3a and V3c an open state to start the transfer processing (Step S13a).

Next, the drive control function 101 pulls the collection piston to collect the target cells C (Step S13b). Specifically, the drive control function 101 moves the collection piston in a direction opposite to the valve V1c. This operation draws the cell suspending solution in the cell suspending solution housing part 17 toward the valve V3c. The drawn cell suspending solution flows in a direction indicated by the arrow Y16 in FIG. 19 and passes in order through the tubes Tug and Tu7, the cell capturing part 19, and the tubes Tu6, Tu4, and Tu3 to move into the collection container SY3.

In this process, the cell suspending solution suspends the target cells C captured in the cell capturing part 19. Thus, the target cells C will move into the collection container SY3 together with the cell suspending solution.

As described above, the cell culture apparatus 1 according to the present embodiment performs sucking from at least either the culture medium housing part 16 or the cell housing container SY1 connected to the supply port 15 into the culture part 11 and discharge from inside the culture part 11 to the discharge path through the sliding of the movable wall 12.

The movable wall 12 moves, whereby the size of the culture area A for the target cells C can be adjusted. When the culture area A increases in size, the cell density of the target cells C decreases, whereas when the culture area A decreases in size, the cell density of the target cells C increases, and thus the cell culture apparatus 1 according to the present embodiment can culture cells at an appropriate cell density. Supply or discharge of the target cells C, the culture medium, and the like can be performed simply by sliding the movable wall 12. Thus, the cell culture apparatus according to the present embodiment can efficiently perform operations related to cell culture. Supply or discharge of the target cells C, the culture medium, and the like is performed in a closed space, whereby it is also possible to prevent microorganisms and other samples from getting mixed.

The culture part 11 is provided in a rotatable manner about the axis of the tubular shape. By rotating the culture part 11 about the axis of the tubular shape during culture, the target cells C move inside the culture part 11, and thus an occurrence of an imbalance can be prevented in the cell density of the target cells in the culture part 11. The target cells C move inside the culture part 11, and thus even if the target cells C move to a part in which the culture medium is absent, they will return to inside the culture medium over time. Thus, the drying of the target cells C can be prevented even if the amount of the culture medium is reduced, and thus a cost reduction can be achieved by reducing the amount of the culture medium for use in culture.

The cell culture apparatus 1 according to the present embodiment performs control to tilt the culture part 11 when discharging the culture medium. Accordingly, the cell culture apparatus 1 according to the present embodiment can discharge the culture medium without applying large force.

The embodiment described above can also be performed after being modified as appropriate by changing part of the configurations or functions of each apparatus. Thus, the following describes a modification according to the embodiment described above as another embodiment. The following mainly describes a point different from the embodiment described above and omits detailed descriptions of points common to the details already described. The modification described below may be implemented individually or implemented in combination as appropriate.

### Modification

The embodiment described above describes a mode in which the drive control function 101 controls the moving mechanism 14 to control the operation of the piston 13. However, the piston 13 may be operated by human hands. According to the cell culture apparatus 1 according to the present modification, work such as culture medium replacement can be performed even in the event of a power failure or the like.

According to at least one embodiment described above, the risk of mixing of microorganisms and the like during cell culture can be reduced and cells can be cultured efficiently at an appropriate cell density.

The term “processor” used in the above description means, for example, a central processing unit (CPU), a graphics processing unit (GPU), or a circuit such as an application specific integrated circuit (ASIC) or a programmable logic device (for example, a simple programmable logic device (SPLD), a complex programmable logic device (CPLD), or a field programmable gate array (FPGA)).

Instead of storing the computer program in the memory, the computer program may be incorporated directly into a circuit of the processor. In this case, the processor reads the computer program incorporated into the circuit and executes it to implement functions. Each processor of the present embodiment is not limited to a case in which each processor is configured as a single circuit, but a plurality of independent circuits may be combined with each other to be configured as one processor to implement its functions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A cell culture apparatus comprising:
a culture medium housing part configured to house a culture medium for culturing cells;
a cell housing part configured to house the cells;
a discharge path for discharge;
a culture part having a hollow tubular shape with holes provided at both ends, the culture medium housing part, the cell housing part, and the discharge path being connected to one of the holes in a switchable manner; and
a movable wall inserted into the culture part from another hole of the culture part and arranged in a slidable manner along an inner peripheral face of the culture part, wherein
the movable wall slides inside the culture part to perform sucking from at least either the culture medium housing part or the cell housing part into the culture part and discharge from inside the culture part to the discharge path.

2. The cell culture apparatus according to claim 1, wherein the movable wall slides inside the culture part in a direction moving away from the one hole to perform sucking from at least either the culture medium housing part or the cell housing part into the culture part and slides inside the culture part in a direction moving close to the one hole to perform discharge from inside the culture part to the discharge path.

3. The cell culture apparatus according to claim 1, wherein the culture part is provided in a rotatable manner about an axis of the tubular shape.

4. The cell culture apparatus according to claim 1, wherein an end provided with the one hole is formed in a tapered shape with a diameter of the tubular shape increasing as a distance from the hole increases.

5. The cell culture apparatus according to claim 4, wherein discharge is performed from inside the culture part to the discharge path by tilting the culture part.

6. The cell culture apparatus according to claim 1, further comprising processing circuitry causing the movable wall to slide in a direction moving away from the one hole to perform sucking from at least either the culture medium housing part or the cell housing part into the culture part and causing the movable wall to slide in a direction moving close to the one hole to perform discharge from inside the culture part to the discharge path.

7. The cell culture apparatus according to claim 6, wherein
the culture part is provided in a rotatable manner about an axis of the tubular shape, and
the processing circuitry performs control to rotate the culture part about the axis of the tubular part.

8. The cell culture apparatus according to claim 6, wherein the processing circuitry performs control to perform discharge from inside the culture part to the discharge path by tilting the culture part.

9. A cell culture method by a cell culture apparatus including: a culture medium housing part housing a culture medium for culturing cells; a cell housing part housing the cells; a discharge path for discharge; a culture part having a hollow tubular shape with holes provided at both ends, the culture medium housing part, the cell housing part, and the discharge path being connected to one of the holes in a switchable manner; and a movable wall inserted into the culture part from another hole of the culture part and arranged in a slidable manner along an inner peripheral face of the culture part, the cell culture method comprising:
performing sucking from at least either the culture medium housing part or the cell housing part into the culture part through sliding of the movable wall in a direction moving away from the one hole; and
performing discharge from inside the culture part to the discharge path through sliding of the movable wall in a direction moving close to the one hole.

10. The cell culture method according to claim 9, wherein
the culture part is provided in a rotatable manner about an axis of the tubular shape, and
the cell culture method further comprises rotating the culture part about the axis of the tubular shape.

11. The cell culture method according to claim 9, further comprising performing discharge from inside the culture part to the discharge path by tilting the culture part.
